(19) United States
(12) Patent Application Publication
(10) Pub. No.: US 2022/0277819 A1
(43) Pub. Date: Sep. 1, 2022

(54) SYSTEMS AND METHOD FOR MONITORING PATIENT TREATMENT

(71) Applicant: SLS HOLDINGS, Little Elm, TX (US)

(72) Inventor: Todd McKeehan, Brookshire, TX (US)

(73) Assignee: SLS HOLDINGS, Houston, TX (US)

(21) Appl. No.: 17/668,056

(22) Filed: Feb. 9, 2022

Related U.S. Application Data
(63) Continuation of application No. 17/511,851, filed on Oct. 27, 2021.
(60) Provisional application No. 63/105,943, filed on Oct. 27, 2020.

(51) Int. Cl. G16H 15/00; G16H 10/60; G16H 40/20

(52) U.S. Cl. CPC G16H 15/00; G16H 10/60; G16H 40/20

(57) ABSTRACT

Systems and methods for monitoring a patient are provided for improving reactions to patient events, auditing, accountability, training tools, hospital compliance, and pharmaceutical compliance, among other things. In an embodiment, a system may include a user device, a server, and a database. Based on a user input by a healthcare practitioner (e.g., the patient's condition, logging the administration of medication to the patient, etc.), a treatment plan may be generated or retrieved from memory (e.g., the database). The treatment plan may, for example, be a schedule that indicates a timing in which the patient must be monitored during the administration of the medication (e.g., where the medication is administered over a 24 hour period) or after the administration of the medication. If the healthcare practitioner completes tasks in accordance with the schedule, the tasks may be logged into memory. If the healthcare practitioner fails to complete tasks in accordance with the schedule, the failure may be logged into memory, communicated to one or more user devices, communicated to an administrator device, and/or communicated to a server that may revise the schedule or generate a new treatment plan.

### CROSS REFERENCE TO RELATED APPLICATIONS

This application claims the benefit of U.S. patent application Ser. No. 17/511,851, filed on Oct. 27, 2021 and U.S. Provisional Patent Application No. 63/105,943, filed on Oct. 27, 2020, the disclosures of which are incorporated by reference herein in their entireties.

### SUMMARY OF THE INVENTION

Embodiments of the disclosed invention allow for one or more healthcare practitioners to record and report various conditions of a patient in a centralized location (e.g., an off premise location), and monitor the patient and practitioners during various timeframes (e.g., at periodic intervals after a patient presents at a healthcare facility). Embodiments of the invention may facilitate communications with other healthcare practitioner in administering treatments to patients.

In an exemplary embodiment, a healthcare practitioner may need to complete the recording or one or more of a patient's vital signs for a particular patient within specific timeframes. Thus, in an embodiment, the invention may involve generating notifications to a healthcare practitioner to record vital signs.

For example, the vital signs may correspond to neurological injury or pathology. Accordingly, such vital signs may correspond to the National Institutes of Health Stroke Scale (NIHSS), the Canadian Neurological Scale (CNS), the European Stroke Scale (ESS), the Glascow Coma Scale (GCS), the Hemispheric Stroke Scale (HSS), the Hunt & Hess Scale, the Mathew Stroke Scale, the Orgogozo Stroke Scale, the Oxfordshire Community Stroke Project Classification, the Scandinavian Stroke Scale, the World Federation of Neurological Surgeons Grading System for Subarachnoid Hemorrhage Scale, or any other medical standard for determining a patient's vital signs.

Accordingly, an embodiment of the invention may act as a reminder and database for healthcare practitioners to complete tasks they perform during the treatment of a patient. As time intervals may vary as a patient's treatment continues and/or the patient's condition progresses, healthcare practitioners may have difficulty knowing the status of the patient's treatment, or at what part of the process the practitioner is at when becoming involved with the patient. This especially occurs when a patient is transferred to a different hospital, department within the hospital, outpatient facilities and/or home health, or any location where a patient may be monitored. Thus, it may be difficult to ascertain which time period the patient should be obtaining the patient's vital signs, what has already been performed, etc. Thus, an embodiment of the invention may aid patient care management by records and generates time stamped information corresponding to the performance of certain healthcare treatments.

In an exemplary embodiment, after infusion of Alteplase (tPA) has begun for a patient, the patient's vital signs may need to be monitored and logged every 15 minutes for the first 15 hours, every 30 minutes for the next 6 hours, and then hourly from the eighth hour on, until 24 hours after the infusion is stopped.

Thus, in an embodiment, a healthcare practitioner may input information corresponding to the patient (e.g., the patient's name, weight, height, baseline vital signs, etc.) in addition to the identity and quantity of the medication being administered (e.g., tPA) into an exemplary system. The system may then determine, based on tables stored in memory, or based on an algorithm using the identity and quantity of the medication along with any other relevant factors as inputs, a schedule associated with monitoring the patient's vital signs post-administration of the medication.

In some embodiments, vital signs may be detected by one or more sensor attached to the patient. For instance, a patient's intracranial pressure may be relevant to a patient's treatment. Accordingly, the system may include an intraventricular catheter, a subdural screw, an epidural sensor, or some other device for monitoring the patient's intracranial pressure. The system may automatically record the intracranial pressure read by the sensing device at a predetermined time, and may either notify one or more healthcare practitioners of the recording, log the recording into memory, alert a healthcare practitioner to attend to the patient (e.g., if a vital sign must be taken when an associated vital sign of the patient meets a predetermined threshold), or some combination thereof.

However, other vital signs may be detected only through observation by a healthcare practitioner. For instance, a patient's Glascow Coma Scale score may be determined through observations of a patient's ability to perform eye movements, speak, and move their body. Thus, an embodiment of the invention may involve notifying a healthcare practitioner of a need to attend to the patient to record the patient's Glascow Coma Scale score at the predetermined time, or in response to related vital sign automatically determined by the system.

In another embodiment, the system may require a combination of data that may be retrieved using one or more sensors in combination with data to be obtained through observation by a healthcare practitioner. Accordingly, in an exemplary embodiment, a system may generate a form on a display device. The form may include inputs corresponding to vital signs to be recorded. If the vital signs may be detected automatically using one or more sensors, those inputs may be automatically populated into the form. Additionally, in order to signal to a healthcare practitioner which data was automatically input into the form (e.g., for when the healthcare practitioner wishes to review the entire form after filling out all data prior to submission of the form) so that a person reviewing the form may ascertain which data was automatically populated by the system, and which data was manually input.

In another embodiment, the system may generate a report. The report may assist a healthcare practitioner and/or administrator in carrying out or improving patient care. For instance, if a patient's scheduled treatments have not been carried out as required, indicators of such failures may be reported to an administrator to facilitate prompt reaction, such as allowing the administrator to coordinate with others to improve the patient's care a priori. The report may include the patient's vital signs, test results, indicators corresponding to assessments and other inputs (or lack thereof) received from one or more healthcare practitioners, medications that have been administered to the patient, etc.

In another embodiment, a healthcare practitioner may be prevented from completing a task at the desired time interval. Accordingly, if the system detects that a task must be completed at a particular time interval, the system may send a communication to the healthcare practitioner, such as by sending a notification to the practitioner's user device. If the healthcare practitioner is unable to complete the task (e.g., the healthcare practitioner is urgently tending to another patient), the healthcare practitioner may react quickly by sending a responsive communication indicating that the task cannot be completed. In response, the system may send a communication to one or more other user devices in the system. The one or more other user devices may belong to one or more other healthcare practitioners who may be better situated to attend to the patient to perform the necessary task, ensuring proper compliance with hospital protocols and no gap in treatment for the patient.

In another embodiment, a system may store a patient's medical records, activities performed by healthcare practitioners in the treatment of a patient, a patient's vital signs, timestamps of the foregoing, and any other relevant information in a database. The system may further include functionality for an administrator's device to view this information. In an embodiment, the system may transmit a communication to the administrator's device periodically, and/or upon the administrator's request (e.g., receiving a communication from the administrator's device to fetch all data or some particular data). This may allow an administrator to audit one or more patients' and/or one or more healthcare practitioners' progress in real time. Thus, embodiments of the present invention may assist in auditing healthcare personnel and policies to increase accountability and improve performance in real-time and in the long term. For instance, an administrator can be informed if a healthcare practitioner is failing to complete tasks, and receive a report with details of such failures (e.g., whether a failure occurred as a result of a shift-change, due to a healthcare practitioner tending to one or more other patient's at the time, due to one healthcare practitioner being assigned to too many patients at a time, etc.). This may allow the administrator to reassign tasks, or perform some other corrective action.

### BRIEF DESCRIPTION OF THE INVENTION

The disclosed invention may further be described in accordance with the following drawings:

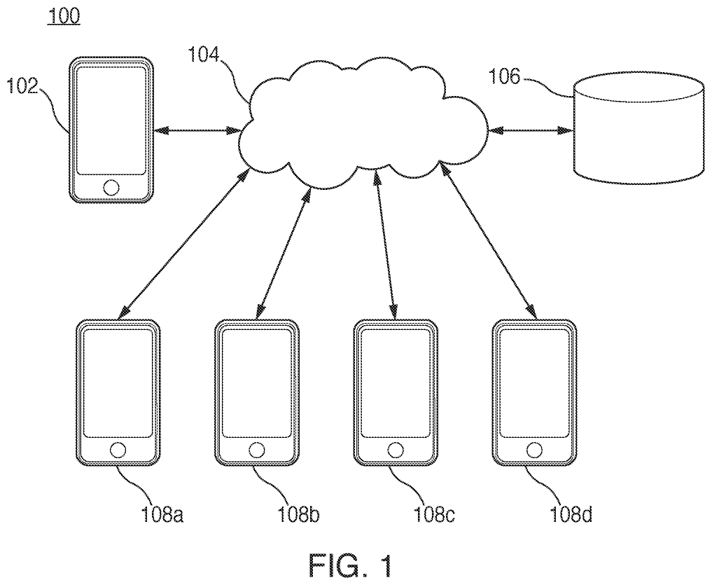

FIG. 1 depicts a system in accordance with embodiments of the invention;

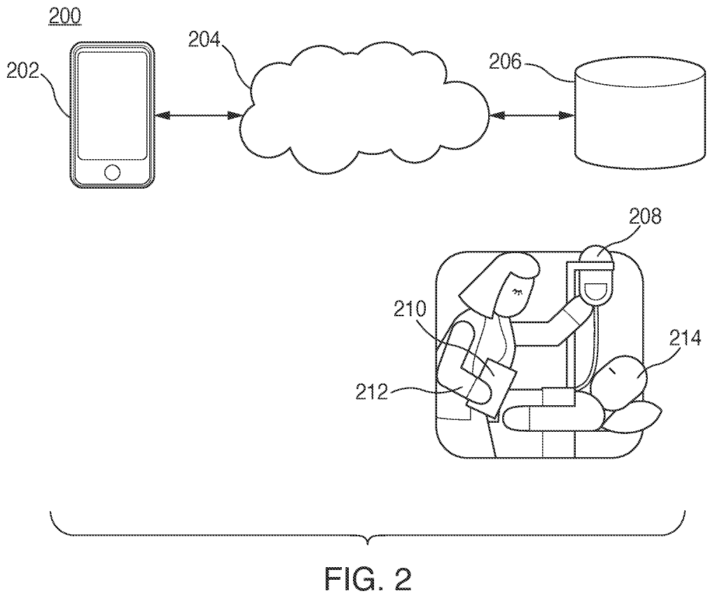

FIG. 2 depicts a system in accordance with embodiments of the invention;

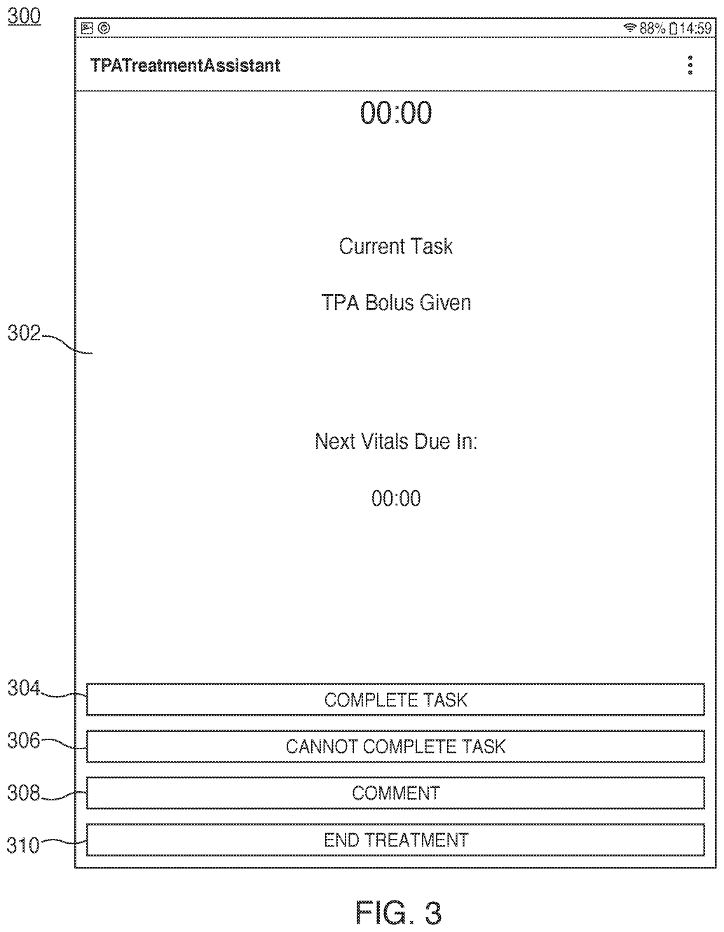

FIG. 3 depicts a user device in accordance with embodiments of the invention;

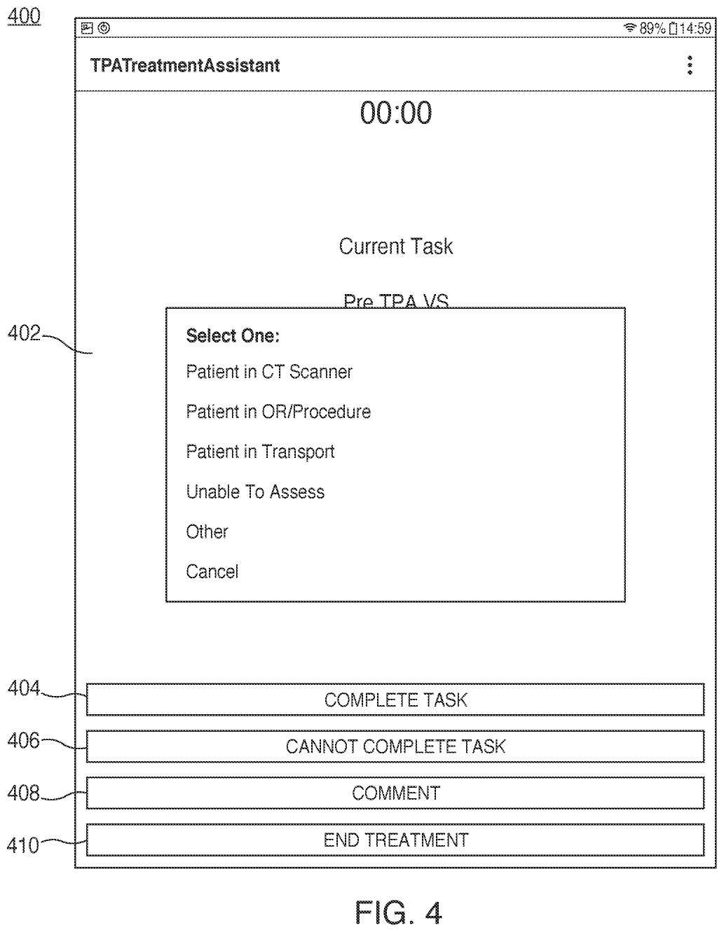

FIG. 4 depicts a user device in accordance with embodiments of the invention;

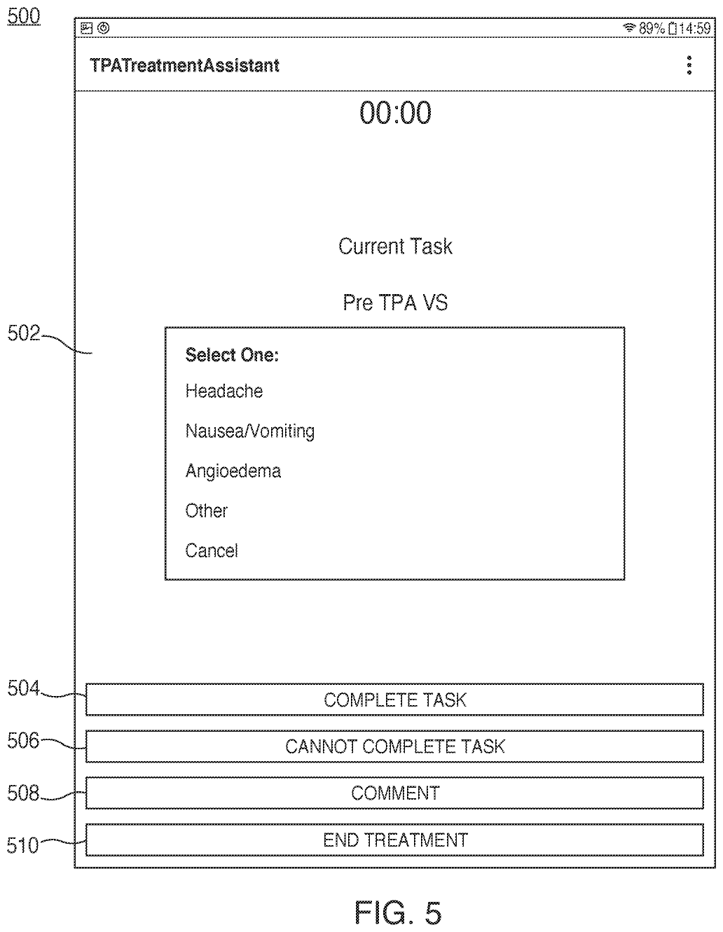

FIG. 5 depicts a user device in accordance with embodiments of the invention;

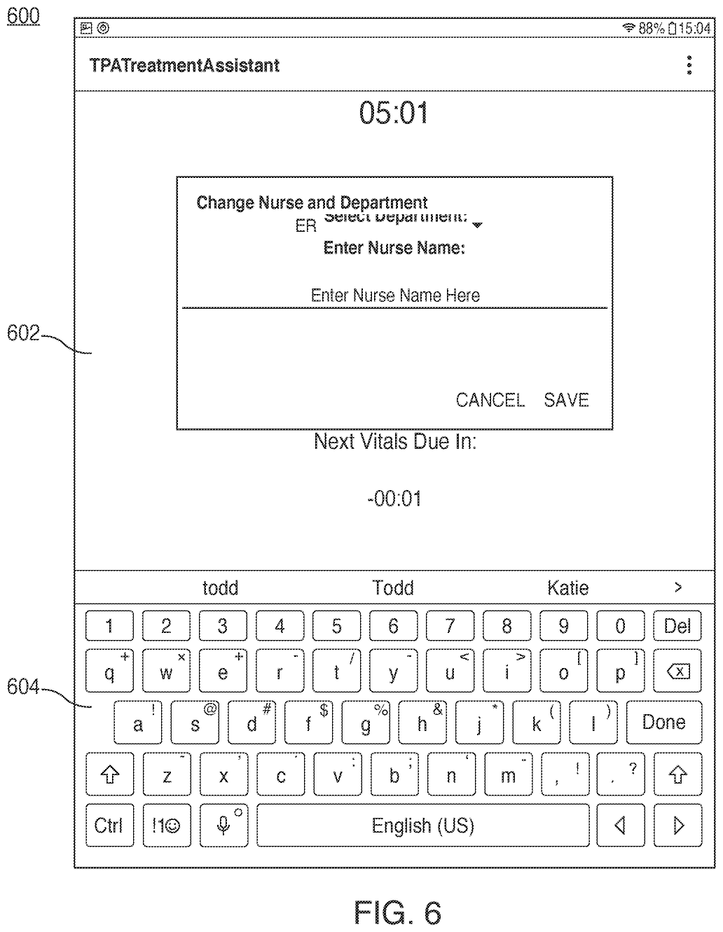

FIG. 6 depicts a user device in accordance with embodiments of the invention; and

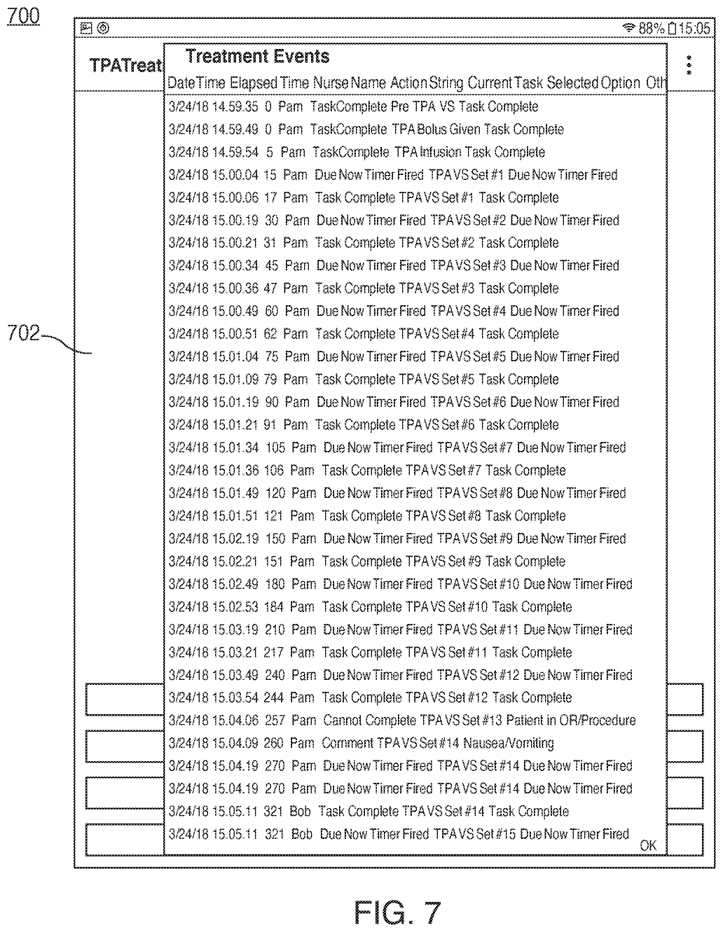

FIG. 7 depicts a user device in accordance with embodiments of the invention; and

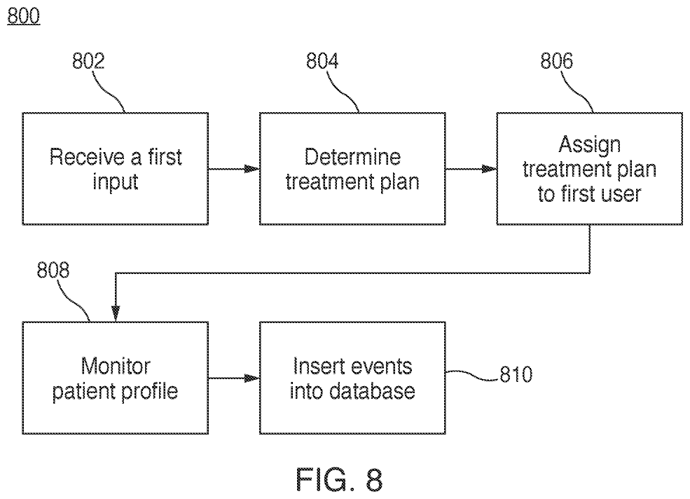

FIG. 8 depicts a method in accordance with embodiments of the invention.

### DETAILED DESCRIPTION OF THE INVENTION

FIG. 1 depicts a system 100 in accordance with an embodiment of the invention. System 100 may include an administrator device 102, a server 104, a database 106, and user devices 108a-d.

Administrator device 102 may be a single device, or one of multiple devices having administrative privileges in system 100. Administrator device may include functionality for viewing database 106 records along with time stamps, tasks that have been completed, when they were completed, etc. This data from database 106 may be received from user devices 108a-d, which may communicate the data to server 104 to enter into database 106.

Administrator device 102 may also communicate with user devices 108a-d directly or through server 104. For instance, administrator device 102 may display to an administrator, via a display screen of administrator device, a report detailing one or more patients' treatment during a hospital stay. For instance, an administrator device 102 may display data corresponding to one or more healthcare practitioners' progress and patient treatment in real time, such as by displaying whether a user (i.e., a healthcare practitioner) is missing periodically assigned assessments or drug administrations. This data may be provided to administrator device 102 via a local application on administrator device 102, or via an electronic mail or other messaging system to administrator device 102 received from server 104 or user devices 108a-d. Administrator device 102 may also display timestamps of actions made by healthcare practitioners (or lack thereof, such as a failure to provide a treatment or assessment at a particular assigned date and/or time). Administrator device may also provide data corresponding to possible reasons for particular events (e.g., a healthcare administrator failing to provide treatment or an assessment for a patient due to a shift change, poor communication between practitioners, a practitioner being busy treating another patient at a particular time, etc.). Administrator device 102 may also display correlations for why similar events occur at a particular frequency (e.g., failures to administer treatment or assessments often occur during shift change, etc.).

Based on an assessment of the report, the administrator may choose to communicate with one or more healthcare practitioners to discuss the results and assessment of the report. Accordingly, the administrator can send a communication from administrator device 102 to any, all, or a combination of user devices 108a-d via server 104 or through direct communications protocols. In another embodiment, administrator device 102 may send a communication to user devices 108a-d, server 104, or another device (e.g., a pharmacy). For instance, administrator device 102 may send a communication to a pharmacy that additional medication is needed for the patient (e.g., based on an assessment of the report). In another embodiment, any of server 104 and user devices 108a-d may send such communications. For instance, if for some reason a supply of a medication being administered to a patient were to reach a low level (or if the healthcare practitioner decides for any other reason that additional medication is needed), user devices 108a may be used to communicate with a pharmacy to order more medication.

Server 104 may be configured to perform much of the analysis performed by system 100. For instance, after a user (e.g., a healthcare practitioner) inputs a patient's information (e.g., the patient's age, height/weight, medical condition, treatment plan, etc.), server 104 may generate a schedule for particular treatments and/or assessments that may be required for optimal results. For instance, server 104 may retrieve from database 106 data corresponding to the patient's condition. The data may further indicate a schedule for treatments and/or assessments relating to that patient's condition. In another embodiment, the user may input a date and/or time at which the user administered a particular treatment to the patient. In some embodiments, the administration of a particular treatment (e.g., tPA) may require periodic assessment of the patient's condition at particular intervals after the administration of the treatment. Accordingly, server 104 may retrieve from the database 106 the schedule pertaining to the particular condition, and may communicate that data to one or more of user devices 108a-d and/or administrator device 102.

Database 106 may be any database capable of storing information pertaining to patient care. Database 106 may be a relational database, a non-relational database, or some other type of database. Database 106 may exist as separate hardware from server 104, may be incorporated into server 104, or may exist in some form in server 104, administrator device 102, and user devices 108a-d. For instance, Each of user devices 108a-d may include a separate local database. Each of user devices 108a-d may periodically or on command synchronize its respective database to the master database 106 (which may be stored within server 104 or separate as its own standalone hardware). In an embodiment, administrator device 102 may determine the frequency at which user devices 108a-d synchronize their databases with database 106, or may send a command to user devices 108a-d to synchronize their databases with database 106 at any time. In order to minimize latency and reduce the potential for lost data, user devices 108a-d may synchronize their databases with database 106 by generating a copy of their individual databases, then transmit those databases to either server 104 or database 106. Server 104 may then transform and load the data received from user devices 108a-d to database 106. In another embodiment, the data may be transformed and loaded into database 106 by database 106 itself. Upon successful completion of merging the data from user devices 108a-108d to database 106, user devices 102 may then receive a communication from server 104 or database 106 to delete the data in order to conserve memory.

User devices 108a-d may be any user device capable of receiving inputs, storing data in memory, and communicating data to another device or server. User devices 108a-d may correspond to any suitable type of electronic device including, but not limited to, desktop computers, mobile computers (e.g., laptops, ultrabooks, etc.), mobile phones, smart phones, tablets, televisions, set top boxes, smart televisions, personal display devices, personal digital assistants (“PDAs”), gaming consoles and/or devices, smart furniture, smart hospital devices, smart vehicles (e.g., ambulance vans, wheelchairs, etc.), wearable devices (e.g., watches), and medical devices (automated medication administering devices, implanted controlled readable medical devices, blood pressure monitors, temperature probes, force sensors, airflow sensors, pacemakers, oximeters, glucometers, electrocardiogram sensors, etc.). In some embodiments, user devices 108a-d may be relatively simple or basic in structure such that no, or a minimal number of, mechanical input options (e.g., keyboard, mouse, trackpad) or touch inputs (e.g., touch screen, buttons) are included. For example, user devices 108a-d may be able to receive and output audio, and may include power, processing capabilities, storage/memory capabilities, and communication capabilities. However, in other embodiments, user devices 108a-d may include one or more components for receiving mechanical inputs or touch inputs, such as a touch screen and/or one or more buttons.

Each of user devices 108a-d may include one or more processors, storage/memory, communications circuitry, one or more microphones or other audio input devices (e.g., transducers), one or more speakers, or other audio output devices, a display screen, one or more optional cameras or other image capturing components. Each of user devices 108a-d may also include one or more medical sensors, including, but not limited to, temperature probes, force sensors, airflow sensors, pressure sensors, pacemakers, oximeters, glucometers, magnetometers, electrocardiogram sensors, heart rate sensors, electroencephalogram sensors, electromyogram sensors, respiration rate sensors, or any other medical device. However, one or more additional components may be included within each of user devices 108a-d, such as a power supply or bus connector, additional input and/or output components, etc.

A user device of user devices 108a-d may include a processor. Such a processor may include any suitable processing circuitry capable of controlling operations and functionality of a user device, as well as facilitating communications between various components within a user device. In some embodiments, a processor may include a central processing unit (“CPU”), a graphic processing unit (“GPU”), one or more microprocessors, a digital signal processor, any other type of processor, or any combination thereof. In some embodiments, the functionality of a processor may be performed by one or more hardware logic components including, but not limited to, field-programmable gate arrays, application specific integrated circuits, application-specific standard products, system-on-chip systems, and/or complex programmable logic devices. Further, one or more processors in user devices 108a-d may include its own local memory, which may store program systems, program data, and/or one or more operating systems. However a processor may run an operating system for a user device of user devices 108a-d, along with (or alternative to) one or more firmware applications, media applications, and/or applications resident thereon. In some embodiments, a processor may run a local client script for reading and rendering content received from one or more websites, server 104, other user devices 108a-d, administrator device 102, or some other device.

A user device of user devices 108a-d may include storage/memory, which may include one or more types of storage mediums such as any volatile or non-volatile memory, or any removable or non-removable memory implemented in any suitable manner to store data for a user device. For example, information may be stored using computer-readable instructions, data structures, and/or program systems. Various types of storage/memory may include, but are not limited to, hard drives, solid state drives, flash memory, permanent memory (e.g., read only memory), electronically erasable programmable read only memory, CD-ROM, digital versatile disk, or other optical storage medium, magnetic cassettes, magnetic tape, magnetic disk storage or other magnetic storage devices, RAID storage systems, or any other storage type or combination thereof. Storage/memory may be implemented as computer readable storage media, which may be any available physical media accessible by processors to execute one or more instructions stored within storage/memory. In some embodiments one or more applications may be run by processors and may be stored in storage/memory.

In some embodiments, storage/memory may include a media system, which may be configured to facilitate communications between user devices 108a-d and administrator device 102, system 104, and/or database 106. For example, the media system may store one or more communications protocols that may be executed by processors for facilitating communications for one or more of user devices 108a-d. In some embodiments a sessions initiation protocol may be used to facilitate media transfer between user devices 108a-d and/or administrator device 102, server 104, and/or database 106. For example, an application layer protocol that is text based may employ real time transport protocol or secure real time transport protocol functions. Other communications may be employed by the media system to support audio, video, and messaging communications. In some embodiments, a web real time communications protocol may be used. In some embodiments, the media system may include instructions that indicate which communications protocol to employ for facilitating media transfer between devices.

A user device of user devices 108a-d may include communications circuitry. Such communications circuitry may include any circuitry allowing or enabling one or more components of a user device to communicate with one another or one or more additional devices (an administrator device 102, one other or more of user devices 108a-d, server 104, or database 106). For example, data corresponding a treatment administered to a patient (e.g., a healthcare practitioner recording into a user device of user devices 108a-d their administering a treatment or performing an assessment of the patient, data received from automated and/or remote monitoring of the patient through one or more sensors, instructions for remotely controlling a medical device and/or medication dispenser) may be communicated over a network, such as the Internet, to server 104 using any number of communications protocols. For example, a network maintaining communications within system 100 may be accessed via Transfer Control Protocol and Internet Protocol (“TCP/IP”) (e.g., any of the protocols used in each of the TCP/IP layers), Hypertext Transfer Protocol (“HTTP”), WebRTC, SIP, and wireless application protocol (“WAP”), or some other type of protocol. In some embodiments devices within system 100 may communicate with one another via a web browser using HTTP. Various additional communication protocols may be used to facilitate communications between devices within system 100 including, but not limited to, Wi-Fi (e.g., 802.11 protocol), Bluetooth, radio frequency systems (e.g., 900 MHz, 1.4 GHz, and 5.6 GHz communication systems), cellular networks (e.g., GSM, AMPS, GPRS, CDMA, EV-DO, EDGE, 3GSM, DECT, IS-136/TDMA, iDen, LTE, or any other suitable cellular network protocol), infrared, BitTorrent, FTP, RTP, RTSP, SSH, and/or VOIP.

Communications circuitry may also include an antenna to facilitate wireless communications with a network using various wireless technologies (e.g., Wi-Fi, Bluetooth, radio frequency, etc.). In another embodiment, one or more devices in system 100 may include one or more universal serial bus (“USB”) ports, one or more Ethernet or broadband ports, and/or any other type of hardware access port.

One or more devices in system 100 may include one or more microphones and/or transducers within or coupled to said one or more devices. One or more devices in system 100 may include one or more speakers within or coupled to said one or more devices. One or more devices in system 100 may also include a display screen, which may also include a touch screen. Various types of displays include, but are not limited to, liquid crystal displays, monochrome displays, color graphics adapter displays, enhanced graphics adapter displays, variable graphics array displays, or another type of display or combination thereof. One or more devices in system 100 may include one or more cameras located within or coupled to the respective device.

In other embodiments, one or more devices in system 100 may include an additional input/output (“I/O”) interface. For example, a device in system 100 may include one or more input components cable of receiving user inputs, such as keyboards, buttons, switches, a mouse, joysticks, or an external controller as an input mechanism for the I/O interface.

FIG. 2 depicts a system 200 in accordance with various embodiments. System 200 may include an administrator device 202, a server 204, a database 206, a medical device 208, and a user device 210 operated by a user/healthcare practitioner 212 tending to a patient 214. System 200 may be substantially similar to system 100.

Medical device 208 may be any medical device capable of administering care to a patient including, but not limited to, administering medicine, sensing and recording vital signs or other conditions of the patient, and communicating corresponding data via a display screen, or to administrator device 202, server 204, user device 210, and/or database 206. For instance, device 208 may be an intraventricular catheter that continuously measures and records a patient's intracranial pressure. Device 208 may also include memory for storing the patient's intracranial pressure, along with communications circuitry for transmitting the corresponding data to user device 210, administrator device 202, server 204, and/or database 206.

Device 208 may work in conjunction with user device 210. For instance, device 208 may measure, record, and transmit vital signs to user device 210 while a healthcare practitioner is performing an assessment of a patient. While the healthcare practitioner is recording results of the assessment into user device 210, device 208 may be providing its own measurements to user device 210. The corresponding data may then be transmitted collectively to administrator device 202, server 204, and/or database 206.

FIG. 3 depicts a user device 300 in accordance with various embodiments. User device 300 may include a touch screen that includes virtual buttons 304, 306, 308, and 310. In an embodiment, a user may record results of a task assigned thereto. For instance, systems such as systems 100 and 200 may generate a treatment plan that includes instructions to perform an assessment of a patient at specific temporal intervals. These instructions may be assigned to one or more healthcare practitioners via their respective user devices. If a particular healthcare practitioner is able to complete a task, the healthcare practitioner may press button 304 indicating that the task may be completed, which may be recorded and/or used for further development of the treatment plan. If the healthcare practitioner cannot complete the task, he or she may press button 306, which may be recorded and/or used for further development of the treatment plan. The healthcare practitioner may also input specific comments via button 308. If the healthcare practitioner determines that the treatment is no longer applicable, the practitioner may also end the treatment via button 310.

FIG. 4 depicts a user device 400 in accordance with various embodiments. User device 400 may, in the event that a healthcare practitioner indicates that he or she cannot complete a task, may display on display screen 402 a form by which the healthcare practitioner may input one or more reasons for being unable to complete the task. For example, a healthcare practitioner may select “Cannot Complete Task” via button 406. A form may appear with options such as “Patient in CT Scanner,” “Patient in OR/Procedure,” “Patient in Transport,” “Unable to Access,” “Other,” or some other option. If the healthcare practitioner selects “Other,” a keyboard may appear that allows the healthcare practitioner to input (of unlimited characters) a specialized reason. In an embodiment, time remaining on a failed task may be added to the next task. For instance, if a healthcare practitioner indicates that they cannot complete a task (e.g., to make an assessment twelve minutes prior to an event), the next temporal interval may be the previously assigned temporal interval, plus the temporal interval corresponding to the failed task (e.g., the next temporal interval if fifteen minutes, and thus the revised temporal interval will be 27 minutes).

Alternatively, in FIG. 5, a healthcare practitioner may complete the task by further inputting data corresponding to an assessment, such as a determination that the patient is suffering from a headache, among other ailments.

In other embodiments, multiple healthcare practitioners may be responsible for the treatment of a patient. In such an embodiment, a healthcare practitioner may sign into a system via user device 600 as shown in FIG. 6. For instance, a healthcare practitioner may input their identity and/or credentials via virtual keyboard 604 shown via display screen 602. In an embodiment depicted in FIG. 7, a display screen 702 may display a report the provides temporal intervals in which treatment (e.g., medication, assessments, etc.) were provided to a patient, and/or are required to be provided to a patient in the future.

FIG. 8 depicts a method in accordance with various embodiments. At step 802, a first input may be received. The input may correspond to a variety of things. For instance, the input may be a form that includes a patient's information (e.g., name, height, weight, medical condition, time of administering medication, a combination of the foregoing, etc.). The input may also correspond to a healthcare practitioner (e.g., a user profile for a particular healthcare practitioner, a time of administering medication and/or performing a particular assessment of the patient, etc.). The input may be a form that includes a large amount of data, may be a form that includes a small amount of data, or may be a single data point.

At step 804, a treatment plan may be determined. In an embodiment, the treatment plan may be a schedule indicating that medication must be administered at precise temporal intervals. The treatment plan may also be a schedule indicating what assessments must be made of the patient's condition at precise temporal intervals. The treatment plan may be a combination thereof. The treatment plan may require specific actions that must be taken by a healthcare practitioner, specific actions that may be automatically performed by an apparatus or device connected to the system (e.g., a measurement and recording of instantaneous blood pressure, images taken of the patient's pupils, etc.), or a combination thereof. The treatment plan may be based on one or more sets of parameters stored in memory (e.g., memory local to a user device or a remote database), stored in memory and in accordance with hospital compliance standards, pharmaceutical compliance standards, or may be input manually by a healthcare practitioner.

At step 806, the treatment plan may be assigned to one or more users. For instance, a schedule of users (e.g., a schedule of work shifts for various healthcare practitioners) may be stored in memory. Based on who is scheduled to work at the healthcare facility in a particular capacity at a particular time, particular treatments may be assigned to one or more healthcare practitioners. For instance, if a first healthcare practitioner has entered the input(s) at step 802, and is scheduled to remain on shift for another 6 hours, and the treatment plan involves assessing the patient's health condition every hour for the next 24 hours, said first healthcare practitioner may be assigned to perform the first 6 hourly assessments. If a second healthcare practitioner is scheduled to work for the next twelve hours, the second healthcare practitioner may be assigned to perform the seventh through eighteenth hourly assessments, and so on. If the patient is transferred to a different medical department, the assignment may automatically be transferred to a healthcare practitioner assigned to the new medical department.

At step 808, the patient profile may be monitored. For instance, during a period preceding a temporal interval in which treatment is assigned to be administered, it may be determined whether a reminder must be communicated to a healthcare practitioner assigned to the task. Accordingly, a communication may be transmitted to a user device corresponding to the healthcare practitioner. Said communication may occur 15 minutes, 30 minutes, 45 minutes, or an hour prior to the assigned task. In another embodiment, the communication may occur at any point prior to or during the temporal interval associated with the assigned task. Communications may be a text message, an email, a push notification, an audio message (e.g., a beep, a verbal communication, etc.), an activation of an application stored in the user device, or some other means for communicating to a user device. The communication may include a simple indicator to perform the assigned task, or may be a report that includes the next assigned task to perform, an assessment of tasks already performed, a patient's prognosis, an identifier for the next healthcare practitioner that will be assigned to perform tasks for the particular patient, or any other data relating to the patient and/or the healthcare practitioner.

In another embodiment, it may be determined that the assigned task is an automated task that can be performed by an apparatus or device connected to a system such as that shown in FIGS. 1 and/or 2. Accordingly, the patient profile may be monitored to determine precisely when the automated task may be performed. In another embodiment, monitoring the patient profile may involve recording vital signs via one or more medical devices (e.g., an EEG monitor, an ECG monitor, an intraventricular catheter, etc.) continuously or within temporal intervals.

At step 810, data corresponding to events may be inserted into a database. For instance, when a healthcare practitioner administers medication, performs an assessment, or provides some other assigned treatment task, such an event may be recorded and inserted into a database. For instance, the healthcare practitioner may input the event into his or her user device, and the user device in turn transmit data corresponding to the event into a server or database located in a separate hardware device. In another embodiment, if a healthcare practitioner fails to provide the assigned treatment during the assigned temporal interval, said failure may be recorded and stored in the database, thereby holding the practitioner accountable for such failures. Other data relating to the failure may also be recorded and stored in the database. For instance, if a healthcare practitioner inputs a note or comment indicating a reason for the failure, said input may be stored in the memory of the healthcare practitioner's user device, the server, or the main database. As another example, if the system automatically detects a potential reason for the failure (e.g., a shift change, the patient being transferred to a different department, the patient being unavailable due to a surgery, a radiology exam, etc.), the reason may be stored in memory. This data may then be stored in memory and retrieved in real time or in the future for the purpose of auditing, training tools, or as raw data for further review, research, analysis, or some other purpose.

In response to the event, other actions may be performed. For instance, if the event represents a failure to provide treatment, other healthcare practitioners may be notified via their own user devices. In another embodiment, an administrator device may be notified. In another embodiment, the treatment plan may be automatically revised accordingly. For instance, if a medication is not administered to the patient, the treatment plan may be revised by increasing or reducing the dosage of medication at different temporal intervals, or the treatment plan may be ended entirely.

The preceding description has been presented only to illustrate and describe exemplary embodiments of the methods and systems of the present invention. It is not intended to be exhaustive or to limit the invention to any precise form disclosed. It will be understood by those skilled in the art that various changes may be made and equivalents may be substituted for elements thereof without departing from the scope of the invention. In addition, many modifications may be made to adapt a particular situation or material to the teachings of the invention without departing from the essential scope. Therefore, it is intended that the invention not be limited to the particular embodiment disclosed as the best mode contemplated for carrying out this invention, but that the invention will include all embodiments falling within the scope of the claims. The invention may be practiced otherwise than is specifically explained and illustrated without departing from its spirit or scope. The scope of the invention is limited solely by the following claims.

What is claimed is:

1. A method comprising:
receiving a first input corresponding to a patient's medical condition;
determining, based on the input, a plurality of temporal intervals corresponding to the patient's condition;
assigning a first temporal interval from the plurality of temporal intervals to a first user;
monitoring the first temporal interval; and
inserting an entry into a database based on an event detected at the first temporal interval.

2. The method of claim 1, wherein the entry represents that the event comprises a second input from the first user, the method further comprising:
receiving the second input from the first user during the first temporal interval, wherein the second input corresponds to a first aspect of the patient's medical condition.

3. The method of claim 2, further comprising:
associating the second input with a user profile representing the first user.

4. The method of claim 1, wherein the entry represents that the event comprises a lack of receipt of an input from the first user during the first temporal interval, the method further comprising:
associating the lack of receipt with a user profile representing the first user.

5. The method of claim 4, further comprising assigning a second temporal interval to at least one of the first user and a second user, wherein the second temporal interval comprises the first temporal interval.

6. The method of claim 1, further comprising:
transmitting a communication to a first user device corresponding to the first user within a second temporal interval preceding the first temporal interval.

7. The method of claim 1, further comprising:
continuously monitoring and recording at least one vital sign of the patient.

8. The method of claim 1, wherein the event represents a communication received from a first user device corresponding to a first user, further comprising:
transmitting the communication to at least one of a second user device.

9. The method of claim 1, further comprising:
modifying, based on the event, a second temporal interval from the plurality of temporal intervals.

10. The method of claim 1, further comprising:
generating a form based on the first input, the form corresponding to a plurality of inputs applicable to the patient's medical condition; and
modifying the form in response to the event.

11. The method of claim 1, further comprising:
generating a report based on data stored in the database; and
transmitting a communication comprising the report to an administrator device.

12. A system, comprising:
a user device configured to:
receive a user input corresponding to a patient; and
monitor the patient via at least one of a sensor and a timer;
a server configured to:
determine, based on the user input, a treatment plan associated with the patient and communicate the treatment plan to the user device; and
a database configured to store data corresponding to the patient.

13. The system of claim 12, wherein each user device comprises a database, and wherein the administrator device is configured to:
transmit instructions to each user device to generate a copy its respective user device database and transmit said copy to the administrator device;
merge each copy with a central database stored in at least one of the administrator device and a storage device; and
upon completion of merging, transmit instructions to each user device to delete data from its respective user device database.

14. The system of claim 12, wherein at least one of the user device and the server is configured to:
detect the occurrence of an event; and
store the event in the database.

15. The system of claim 14, wherein the server is further configured to modify the treatment plan based on the detected occurrence.

16. The system of claim 12, wherein the user device is further configured to transmit at least one record data measured by the sensor to at least one of the server and the database.

17. The system of claim 16, wherein upon receipt of the record data, the sensor is further configured to:
record the data into the database; and
at least one of:
receive data corresponding to a second treatment plan in response to recording the data into the database; and
modify the first treatment plan based on the record data.

18. A system, comprising:
a user device configured to:
receive a user input corresponding to a patient;
determine, based on the user input, a treatment plan associated with the patient; and
monitor the patient via at least one of a sensor and a timer;
a server configured to:
receive record data from the user device, said data corresponding to a data input received from the at least one sensor and timer;
at least one of:
modify the treatment plan based on the record data; and
transmit the record data to a database; and
a database configured to receive the record data.

19. The system of claim 18, wherein the server is further configured to receive a second treatment plan from the database based on the record data.

20. The system of claim 18, wherein at least one of the user device and the server is configured to generate a report corresponding to the treatment plan.
